(19) United States
(12) Patent Application Publication
(10) Pub. No.: US 2009/0280507 A1
(43) Pub. Date: Nov. 12, 2009

(54) METHOD FOR MEASUREMENT OF SARS VIRUS NUCLEOCAPSID PROTEIN, REAGENT KIT FOR THE MEASUREMENT, TEST DEVICE, MONOCLONAL ANTIBODY DIRECTED AGAINST SARS VIRUS NUCLEOCAPSID PROTEIN, AND HYBRIDOMA CAPABLE OF PRODUCING THE MONOCLONAL ANTIBODY

(75) Inventors: Kotaro Fujimoto, Kobe-shi (JP); Tadahiro Kajita, Abiko-shi (JP); Kazuhiko Takeda, Kobe-shi (JP); Takashi Okamoto, Nagoya-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi, Hyogo (JP)

(21) Appl. No.: 12/090,055

(22) PCT Filed: Oct. 11, 2006
(86) PCT No.: PCT/JP2006/320330

§ 371 Date: Apr. 11, 2008

(30) Foreign Application Priority Data
Oct. 11, 2005 (JP) 2005-296542

(51) Int. Cl. G01N 33/573; G01N 33/536; G01N 33/543; C07K 16/08; C12N 5/16

(52) U.S. Cl. 435/7.9; 436/536; 436/518; 530/388.3; 435/339

(57) ABSTRACT

The present invention provides a method for measuring SARS virus nucleocapsid protein (SARS-NP) using first and second antibodies both binding specifically to SARS-NP, wherein the first or second antibody is an antibody recognizing an epitope located in a region (Region C) of amino acid 283 to 422 from the N-terminus of the amino acid sequence of SARS-NP.

### TECHNICAL FIELD

The present invention relates to a method, reagent kit and test device for measuring SARS virus nucleocapsid protein (SARS-NP). Further, the present invention relates to a monoclonal antibody directed to SARS-NP and a hybridoma which produces the monoclonal antibody.

### BACKGROUND ART

Severe Acute Respiratory Syndrome (SARS) is an infectious disease which has been discovered recently. It is demonstrated that the causative factor of SARS is new virus which is classified into Coronavirus. The known diagnostic method of SARS infection is a method of detecting SARS virus in a sample by using an immunological measurement. Such method includes the methods described in Susanna K. P. Lau, Patrick C. Y. Woo, Beatrice H. L. Wong, Hoi-Wah Tsoi, Gibson K. S. Woo, Rosana W. S. Poon, Kwok-Hung Chan, William I. Wei, J. S. Malik Peiris, and Kwok-Yung Yuen, “Detection of Severe Acute Respiratory Syndrome (SARS) Coronavirus Nucleocapsid Protein in SARS Patients by Enzyme-Linked Immunosorbent Assay”, Journal of Clinical Microbiology, Vol. 42, No. 7, P. 2884-2889; and Xiao-yan Che, Li-wen Qiu, Yu-xian Pan, Kun Wen, Wei Hao, Li-ya Zhang, Ya-di Wang, Zhi-yong Liao, Xu Hua, Vincent C. C. Cheng, and Kwok-yung Yuen, “Sensitive and Specific Monoclonal Antibody-Based Capture Enzyme Immunoassay for Detection of Nucleocapsid Antigen in Sera from Patients with Severe Acute Respiratory Syndrome”, Journal of Clinical Microbiology, Vol. 42, No. 6, P. 2629-2635.

Susanna K. P. Lau, et al., supra, describe a measurement method of using enzyme-linked immunoassay (ELISA) with polyclonal antibodies directed against SARS-NP. Specifically, in this method, polyclonal antibodies are immobilized on an ELISA plate to which a sample and labeled polyclonal antibodies are added successively to form complexes and detect them.

Xiao-yan Che, et al., supra, describe a measurement method by ELISA using monoclonal and polyclonal antibodies respectively directed against SARS-NP. Specifically, 3 types of monoclonal antibodies are immobilized on an ELISA plate to which a sample and labeled polyclonal antibodies are added successively to the plate to form complexes and detect them.

The methods described by Susanna K. P. Lau, et al. and by Xiao-yan Che, et al. are based on ELISA. Generally, ELISA is said to be a relatively highly sensitive measurement method among immunological measurement methods. In diagnosis of virus infection, on the other hand, a highly rapid and easy immunochromatographic method is frequently used. However, the immunochromatographic method has lower sensitivity than ELISA, and even if the polyclonal and monoclonal antibodies described by Susanna K. P. Lau, et al. or Xiao-yan Che, et al. are applied to the immunochromatographic method, there is a possibility that sufficient sensitivity cannot be obtained.

### DISCLOSURE OF THE INVENTION

### Problems to be Solved by the Invention

An object of the present invention is to provide a method of measuring SARS-NP with higher sensitivity than conventional, which can be applied not only to a measurement method such as ELISA that is relatively highly sensitive among immunological measurement methods but also to immunochromatography that is relatively low sensitive among immunological measurement methods, as well as a reagent kit, a test device, a monoclonal antibody, and a hybridoma producing the monoclonal antibody, which are used in the measurement.

### Means for Solving the Problems

In view of the above problems, the present invention provides a method for measuring SARS virus nucleocapsid protein (SARS-NP) using first and second antibodies both binding specifically to SARS-NP, wherein the first or second antibody is an antibody recognizing an epitope located in a region (Region C) of amino acid 283 to 422 from the N-terminus of the amino acid sequence of SARS-NP.

The present invention also provides a reagent kit for measuring SARS virus nucleocapsid protein (SARS-NP) using first and second antibodies both binding specifically to SARS-NP, which comprises a combination of a solid phase to which the first antibody is immobilized and a reagent containing the second antibody labeled with a labeling substance. The first and second antibodies are antibodies binding specifically to SARS-NP. The first or second antibody is an antibody recognizing an epitope located in a region (Region C) of amino acid 283 to 422 from the N-terminus of an amino acid sequence of SARS-NP.

Further, the present invention provides an immunochromatographic test device for measuring SARS virus using first and second antibodies both binding specifically to SARS virus nucleocapsid protein (SARS-NP), wherein:

the first antibody is immobilized on a solid phase, and the second antibody is labeled with a labeling substance,

the immunochromatographic test device comprises a sample addition part to which a measurement sample is added and a sample developing part in which the measurement sample added to the sample addition part is developed; the sample developing part having a judging part to which the first antibody is immobilized and the measurement sample added to the sample addition part being developed toward at least the judging part, and

the first or second antibody is an antibody recognizing an epitope located in a region (Region C) of amino acid 283 to 422 from the N-terminus of the amino acid sequence of SARS-NP.

The present invention also provides a monoclonal antibody which binds specifically to SARS virus nucleocapsid protein (SARS-NP) and is produced by a hybridoma having an accession number of FERM ABP-10678.

The present invention also provides a monoclonal antibody which binds specifically to SARS virus nucleocapsid protein (SARS-NP) and is produced by a hybridoma having an accession number of FERM ABP-10679.

The present invention also provides a monoclonal antibody which binds specifically to SARS virus nucleocapsid protein (SARS-NP) and is produced by a hybridoma having an accession number of FERM ABP-10680.

The present invention also provides a monoclonal antibody which binds specifically to SARS virus nucleocapsid protein (SARS-NP) and is produced by a hybridoma having an accession number of FERM ABP-10686.

The present invention also provides a monoclonal antibody which binds specifically to SARS virus nucleocapsid protein (SARS-NP) and is produced by a hybridoma having an accession number of FERM ABP-10687.

The present invention also provides a hybridoma which is deposited under an accession number of FERM ABP-10678.

The present invention also provides a hybridoma which is deposited under an accession number of FERM ABP-10679.

The present invention also provides a hybridoma which is deposited under an accession number of FERM ABP-10680.

The present invention also provides a hybridoma which is deposited under an accession number of FERM ABP-10686.

The present invention also provides a hybridoma which is deposited under an accession number of FERM ABP-10687.

### EFFECTS OF THE INVENTION

According to the measurement method of the present invention, SARS-NP can be measured with higher sensitivity than the prior art methods. SARS virus can thereby be detected easily with high sensitivity.

### BRIEF DESCRIPTION OF THE DRAWINGS

FIG. 1 schematically illustrates one embodiment of a test device used in immunochromatography using the monoclonal antibodies of the present invention.

FIG. 2 schematically illustrates one embodiment of a test device used in immunochromatography using the monoclonal antibodies of the present invention.

FIG. 3 schematically illustrates an amino acid sequence of SARS-NP and 3 regions (Regions A, B and C) contained therein.

FIG. 4 shows the results of Example 3.

FIG. 5 shows the results of Example 4.

FIG. 6 shows the results of Example 5.

### BEST MODE FOR CARRYING OUT THE INVENTION

In the present invention, SARS-NP is measured by an immunological method. Specifically, SARS-NP is measured by forming a complex consisting of SARS-NP, the first antibody binding specifically to SARS-NP and the second antibody binding specifically to SARS-NP. The present inventors made extensive study, focusing attention on the specificity of antibodies used in such measurement and on a combination thereof, and as a result they arrived at completion of the present invention.

It is said that a nucleoprotein SARS-NP relatively hardly undergoes mutation. In the measurement method of the present invention, therefore, antibodies binding specifically to SARS-NP are used as the first and second antibodies to measure SARS-NP.

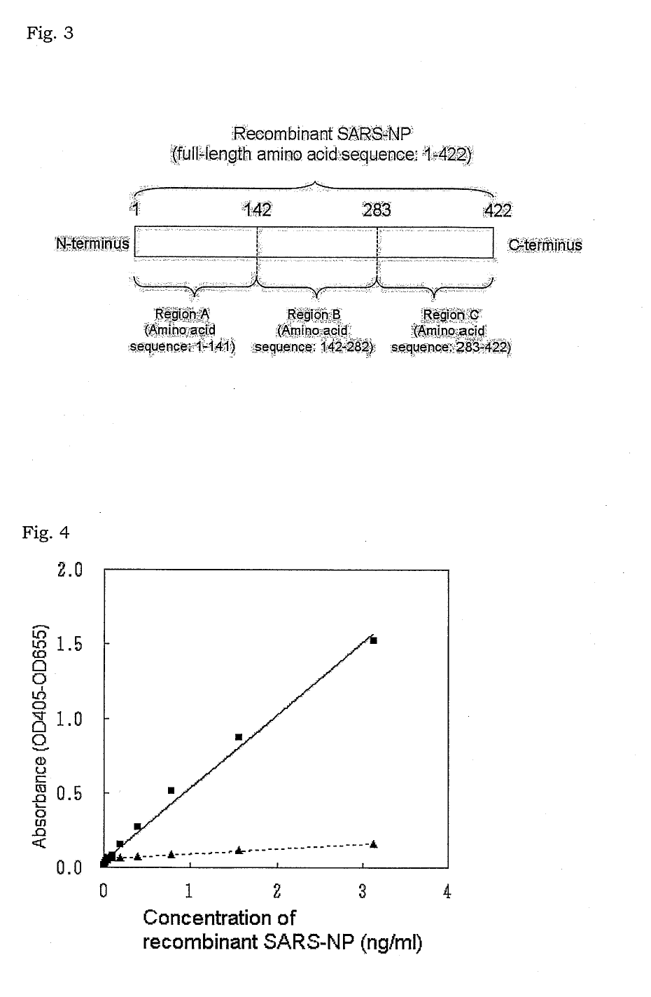

An amino acid sequence (full length: 422 residues) of a nucleocapsid protein derived from SARS TOR2 strain is shown in Gen Bank (Accession No. AY274119; Protein ID: AAP41047.1). In the present invention, this amino acid sequence of SARS-NP was divided into 3 regions as shown in FIG. 3, and a plurality of antibodies each recognizing an epitope located in each region were obtained. Then, various combinations of these antibodies were used to measure SARS-NP. It was thereby found that highly sensitive measurement could be achieved by using, as the first or second antibody, an antibody recognizing an epitope located in the region (Region C) of amino acid 283 to 422 from the N-terminus of the amino acid sequence of SARS-NP.

Preferable examples of the combination of the first and second antibodies include a combination of an antibody recognizing an epitope located in a region (Region A) of amino acid 1 to 141 from the N-terminus of the amino acid sequence of SARS-NP and an antibody recognizing an epitope located in the Region C and a combination of an antibody recognizing an epitope located in a region (Region B) of amino acid 142 to 282 from the N-terminus of the amino acid sequence of SARS-NP and an antibody recognizing an epitope located in the Region C.

When the first antibody is immobilized on the solid phase, the first antibody is preferably an antibody recognizing an epitope located in the Region A or an antibody recognizing an epitope located in the Region C. When the first antibody is immobilized on the solid phase, the combination of the first and second antibodies is preferably a combination wherein the first antibody is an antibody recognizing an epitope present in Region A or an antibody recognizing an epitope located in the Region B and the second antibody is an antibody recognizing an epitope located in the Region C. Another combination includes a combination wherein the first antibody is an antibody recognizing an epitope located in the Region C and the second antibody is an antibody recognizing an epitope located in the Region A or an antibody recognizing an epitope located the in Region B.

It is not necessary that the amino acid sequence of SARS-NP used in the present invention is completely identical to the amino acid sequence of SARS-NP disclosed in Gen Bank (Accession No. AY274119; Protein ID: AAP41047.1). The amino acid sequence of SARS-NP used in the present invention may contain a deletion, substitution or addition of some amino acids from or to the amino acid sequence of SARS-NP disclosed in Gen Bank (Accession No. AY274119; Protein ID: AAP41047.1).

The first and second antibodies may be polyclonal and/or monoclonal antibodies. From the viewpoint of specificity, either of the first or second antibody is preferably a monoclonal antibody, and most preferably, both of the first and second antibodies are monoclonal antibodies.

The immunological measurement method includes, for example, turbitometric immunoassay (TIA), nephelometric immunoassay (NIA), latex agglutination immunoassay (LIA), radioimmunoassay (RIA), enzyme immunoassay (EIA) or enzyme linked immunosorbent assay (ELISA), fluorescent immunoassay (FIA) and chemiluminescent immunoassay (CLIA). Another example includes an immunochromatographic method using a test device comprising a membrane carrier to which an antibody is immobilized. As an immunological measurement method in diagnosis of viral infection, immunochromatography is preferable from the viewpoint of rapidness and easiness. For preventing contamination caused by contacting with a virus-containing sample, the measurement is preferably automated. From the viewpoint of the automatization, sensitivity and general versatility of the measurement, ELISA is preferable as an immunological measurement method.

Depending on the measurement method, an antibody may be labeled with a labeling substance or immobilized on a carrier.

The labeling substance for labeling the antibody is suitably selected depending on the measurement method. For example, when the measurement method is RIA, the labeling substance includes radioisotopes such as ¹²⁵I, ¹⁴C and ³²P. The labeling substance in EIA or ELISA includes enzymes such as β-galactosidase, peroxidase and alkaline phosphatase. The labeling substance in FIA includes fluorescent dyes such as fluorescein derivatives and rhodamine derivatives. The labeling substance in CLIA includes chemiluminescent substances such as luminol, isoluminol, and acridinium derivatives. The labeling substance in immunochromatography includes gold colloid, colored latex particles and fluorescent latex particles.

The carrier for immobilizing an antibody is not particularly limited insofar as it has high binding property for the antibody, and examples of such carriers include synthetic organic polymer compounds such as polyvinyl chloride, polyvinylidene fluoride (PVDF), polystyrene, a styrene-divinyl benzene copolymer, a styrene-maleic anhydride copolymer, nylon, polyvinyl alcohol, polyacrylamide, polyacrylonitrile and polypropylene; polysaccharides such as dextran derivatives, agarose gel and cellulose; and inorganic polymer compounds such as glass, silica gel and silicone. These materials may be those into which functional groups such as an amino, aminoacyl, carboxyl, acyl, hydroxyl or nitro group has been introduced. The shape of the carrier may be selected suitably depending on the measurement method used and includes flat-shaped (e.g., a microtiter plate (ELISA plate) and a disk), particle (e.g., beads), tubular (e.g., a test tube and a tube), fibrous and membrane-shaped. As the method of immobilizing the SARS-NP antibody on the carrier, methods known in the art, such as a physical adsorption method, an ionic bonding method, a covalent bonding method and an inclusion method, may be used.

A reagent containing the antibodies used in the measurement method may be in the form of a solution depending on the measurement method. In this case, the reagent may comprise known components in combination with the antibodies. That is, the known components such as a buffering agent for giving appropriate pH for the antibody-antigen reaction, a reaction enhancer for promoting the antibody-antigen reaction, a reaction stabilizer and blocker for inhibiting non-specific reaction, a preservative for improving the storage stability of the reagent, etc may be combined with the antibodies.

A measurement sample used in the measurement method is a sample that may possibly contain SARS virus or a solution obtained by treating the sample with a buffer, and is not particularly limited insofar as it does not inhibit the measuring reaction. The sample includes, for example, body fluids such as blood, serum, nasal discharge, sputum and a pharyngeal swab.

Hereinafter, the immunochromatography to which the measurement method of the present invention is applied is described.

A typical example of the immunochromatographic method is a method in which a substance to be measured, the first antibody immobilized on the carrier, and the second antibody labeled with the labeling substance are reacted to form a complex consisting of the substance to be measured, the first antibody and the second antibody on the carrier, and the labeling substance of the second antibody is detected, whereby the complex is detected or quantified. Such immunochromatography includes flow-through type and lateral-flow type. The flow-through type immunochromatography is a method in which a solution containing a substance to be measured is passed through vertically to a membrane carrier to which the first antibody is immobilized. On the other hand, the lateral flow type immunochromatography is a method in which a solution containing a substance to be measured is developed horizontally to a membrane carrier to which the first antibody is immobilized.

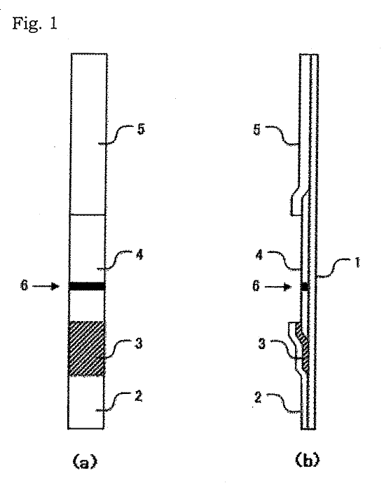

FIG. 1 is a schematic diagram of the test device of lateral flow type. FIGS. 1 (a) and (b) are plane and side views of the test device, respectively. As shown in FIG. 1, the test device of lateral flow type comprises, on a substrate 1 having an adhesive layer thereon, a sample addition member 2, a label-retaining member 3, a chromatographic membrane carrier 4 and an absorbent member 5. The label-retaining member 3 is provided in contact with the sample addition member 2 and retains the second antibody labeled with the labeling substance. The chromatographic membrane carrier 4 is provided in contact with the label-retaining member 3 and has a judging part 6 to which the first antibody is immobilized. The absorbent member 5 is provided in contact with the chromatographic membrane carrier 4.

In one embodiment of the present invention, a measurement sample, when dropped onto the sample addition member 2 in FIG. 1, moves by capillary phenomenon through the sample addition member 2, the label-retaining member 3, the chromatographic membrane carrier 4 and the absorbent member 5 in this order. When a substance to be measured is contained in the measurement sample, this substance reacts with the second antibody in the label-retaining member 3 to form a complex. The complex is captured by the first antibody immobilized on the judging part 6 in the chromatographic membrane carrier 4. As shown in FIG. 1, a band of the labeling substance of the second antibody thereby appears on the judging part 6, thus enabling the visual detection of the substance to be measured.

The chromatographic membrane carrier 4 may further comprise, downstream from the judging part 6, a control part for confirming that the dropped measurement sample has passed through the judging part 6. For example, in case where biotin is immobilized on this control part and avidin which binds to biotin is labeled with a labeling substance and retained on the label-retaining member 3, the avidin in the label-retaining member 3, together with the measurement sample, migrate on the chromatographic membrane carrier 4. The avidin is not captured by the second antibody in the judging part 6 but captured by the biotin in the control part. A band of the labeling substance of the avidin thereby appears in the control part. Because the control part is provided downstream from the judging part 6, it can be confirmed by observing this band that the sample has passed through the judging part 6. Alternatively, avidin may be immobilized on the control part and biotin labeled with a labeling substance may be retained on the label-retaining member 3. The substances to be immobilized on the control part and to be retained on the label-retaining member 3 may be not a combination of avidin and biotin. It is noted that the substance retained in the label-retaining member 3 is the substance that does not react with the substance to be measured or the second antibody immobilized on the judging part.

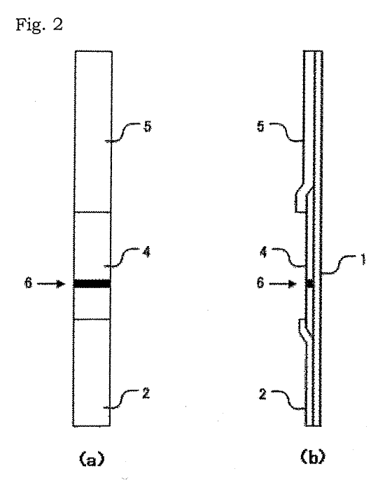

As shown in FIG. 2, the test device may be the one not having the label-retaining member. In this case, the second antibody is previously mixed with a sample to prepare a measurement sample, and this measurement sample can be dropped onto the sample addition member 2 in the test device.

The present invention can be applied to an immunochromatographic detection kit for detecting SARS virus, which comprises the detection device described above. Such kit can comprise, for example, a pretreatment solution for treating a sample to prepare a measurement sample, the test device, a reagent containing various antibodies, etc.

Hereinafter, the ELISA to which the measurement method of the present invention is applied is described.

In ELISA, a microplate such as an ELISA plate to which an antibody or antigen directed to the substance to be measured is immobilized is used. For example, in case where the substance to be measured is an antigen, a microplate to which the first antibody directed to the substance to be measured is immobilized is used. First, the measurement sample is added to the microplate, and the substance to be measured in the measurement sample is allowed to form a complex with the immobilized first antibody. Then, the second antibody labeled with the labeling substance such as enzyme is added thereto, to form a complex consisting of the immobilized first antibody, the substance to be measured and the labeled second antibody on the microplate. Thereafter, the labeling substance of the second antibody in the complex is utilized to detect or quantify the substance to be measured.

The labeling substance for labeling the second antibody includes enzymes such as peroxidase, galactosidase and alkali phosphatase.

Accordingly, the present invention can be applied to an ELISA detection kit for detecting SARS virus, which comprises the microplate described above. Such kit can comprise, for example, the microplate to which the first antibody is immobilized, a washing solution for washing each well of the microplate, a substrate for an enzyme that labels the second antibody, a reagent containing various antibodies, etc. The washing solution includes buffers at predetermined salt concentrations.

Hereinafter, a method of preparing the monoclonal antibody is described. The monoclonal antibody can be prepared according, for example, to the method described in Koehlar and Milstein, Nature 256, 495-497, 1975. That is, the monoclonal antibody can be produced as follows: spleen cells obtained from an animal immunized with the antigen are fused with myeloma cells, and among the resulting fused cells (hereinafter referred to as hybridoma), the cell producing a specific antibody to the antigen is selected, and this hybridoma is cultured in a large amount or proliferated in the peritoneal cavity of an animal, and from the culture or ascites, the monoclonal antibody can be separated. Hereinafter, the method of producing a monoclonal antibody directed to SARS-NP is described in the following [I] to [V]:

### [I] Antigen

The antigen used in preparation of the monoclonal antibody directed against SARS-NP can be obtained by purification of a SARS virus-containing sample. The SARS virus-containing sample includes, for example, blood collected from SARS patients and culture fluids obtained by intentionally culturing SARS virus. Alternatively, the antigen can be obtained by genetic engineering techniques.

### [II] Immunization Step

Purified SARS-NP, recombinant SARS-NP obtained by genetic engineering techniques, or a partial peptide thereof is dissolved or suspended in an appropriate buffer such as a phosphate buffer and is used as an antigen solution. Generally, the antigen solution may be prepared so as to have an antigen concentration of about 50 to 500 μg/mL. Peptide antigens or the like that have low antigenicity can be linked to an appropriate carrier protein such as albumin and keyhole limpet hemocyanin (KLH) to be used.

Animals to be immunized with the antigen (hereinafter referred to as animals to be immunized) include mammals such as mouse, rat, hamster, horse, goat, and rabbit. The animals to be immunized are preferably rodents, in particular mouse.

Immunization can be carried out by administering an antigen solution to an animal to be immunized by subcutaneous, intracutaneous, intraperitoneal or intravenous injection or the like. In order to enhance the response of the animal to be immunized to the antigen, the antigen solution can be administered as a mixture with an adjuvant. The adjuvant is a substance which does not work as an antigen, but upon administration with an antigen, can enhance the immune response of the animal to be immunized to the antigen. The usable adjuvant includes Freund complete adjuvant (FCA), Freund incomplete adjuvant (FIA), Ribi (MPL), Ribi (TDM), Ribi (MPL+TDM), Bordetellapertussis vaccine, muramyl dipeptide (MDP), aluminum adjuvant (ALUM) and a combination thereof.

Preferably, FCA is used in the primary immunization with an antigen solution to the animal to be immunized and FIA or Ribi adjuvant is used in the booster immunization.

Specific example of the immunization method is as follows. For example, when a mouse is used as the animal to be immunized, 0.05 to 1 mL of the antigen solution (10 to 200 μg antigen) mixed with an adjuvant is injected intraperitoneally, subcutaneously, intramuscularly or via a tail vein into the mouse. Booster immunization is carried out 1 to 4 times on every about 4 to 21 days after the primary immunization. About 1 to 4 weeks after the booster immunization, final immunization is carried out. In the final immunization, an adjuvant-free antigen solution is preferably used. About 3 to 5 days after the final immunization, spleen cells are obtained from the immunized animal. The spleen cells thus obtained are antibody-producing cells.

### [III] Cell Fusion Step

In this step, the spleen cells obtained from the immunized animal are fused with myeloma cells to prepare a hybridoma. As the myeloma cells, those derived from a mouse, rat, human etc. are used, and examples of such cells include established myeloma cells such as mouse myeloma P3X63-Ag8, P3X63-Ag8-U1, P3NS1-Ag4, SP2%-Ag14 and P3X63-Ag8.653. Some myeloma cells produce an immunoglobulin light chain, and when such myeloma cell is used in cell fusion, this light chain may randomly link to an immunoglobulin heavy chain produced by the spleen cell. Accordingly, a myeloma cell not producing an immunoglobulin light chain, for example, P3X63-Ag8.653 or SP2%-Ag14 is preferably used. The spleen cells and myeloma cells are preferably those derived from the animals of the same species, particularly of the same strain. The storage of the myeloma cells may be carried out according to a method known per se, for example, by subculturing the cells in a general medium to which horse, rabbit or bovine fetal serum is added, and freezing the obtained culture. For cell fusion, cells in a logarithmic growth phase are preferably used.

The method of producing a hybridoma by fusion of spleen cells with myeloma cells can be exemplified by a method in which polyethylene glycol (PEG) is used, a method in which Sendai virus is used and a method in which an electric fusion apparatus is used. In the PEG method, for example, spleen cells and myeloma cells with a mixing ratio of 1:1 to 10:1, preferably of 5:1 to 10:1, may be suspended in a suitable medium or a buffer containing about 30 to 60% PEG (average molecular weight 1,000 to 6,000) and incubated for about 30 seconds to 3 minutes under the conditions of a temperature of about 25 to 37° C. and pH of 6 to 8. After the incubation is finished, the cells are washed to remove the PEG solution, suspended again in the medium, then seeded in a microtiter plate to continue the culture.

### [IV] Selection of Hybridoma

The cells after the fusion procedure are cultured in a selective medium to select the hybridoma. The selective medium is a medium in which the parent cell strain is killed and only the hybridoma can grow. Usually, a hypoxanthine-aminopterin-thymidine medium (HAT medium) is used. Selection of the hybridoma is generally carried out by exchanging a part of the medium, preferably about half of the medium, with the selective medium after 1 to 7 days of the fusion procedure, and then culturing the cells while the medium is exchanged repeatedly every 2 to 3 days in the same manner. The well in which a hybridoma colony grows is confirmed by observation under a microscope.

Whether the growing hybridoma produces a desired antibody or not can be confirmed by collecting a culture supernatant and performing an antibody titer assay by a conventional method. For example, the above supernatant diluted serially is added to, and reacted with, an antigen protein immobilized on a carrier, and further reacted with a secondary antibody (anti-globulin antibody, anti-IgG antibody, anti-IgM antibody etc.) labeled with a fluorescence substance, an enzyme, or a radioisotope (RI), whereby the antibody produced into the supernatant can be detected and the antibody titer can be measured. When the antigen is for example an enzyme, the enzyme is reacted with the above supernatant and then reacted with a suitable substrate to determine an activity of inhibiting the enzyme, whereby the antibody can be detected and the antibody titer can be measured. By screening the culture supernatant in each well of the plate in this manner, the hybridoma producing the desired antibody is obtained.

Then, a single clone is isolated by a limiting dilution method, a soft agar method, a method in which a fluorescence-activated cell sorter is used, etc. In the limiting dilution method, for example, a hybridoma colony is diluted serially with a medium so as to culture the hybridoma with about 1 cell/well, whereby a hybridoma clone producing the desired antibody can be isolated. When the resulting antibody-producing hybridoma clone is frozen in the presence of a cryoprotective agent such as approx. 10 v/v % of dimethyl sulfoxide (DMSO), glycerin or the like and stored at −1.96° C. to −70° C., the hybridoma clone can be stored for half a year or semi-permanently. The cells are rapidly thawed in a thermostatic bath at about 37° C. at use and then immediately used. The cells are used preferably after sufficient washing such that the cytotoxicity of the cryoprotective agent does not remain.

For examining the immunoglobulin subclass of the antibody produced by the hybridoma, the hybridoma is cultured under general conditions, and the antibody secreted into the culture supernatant may be analyzed by using a commercially available kit for determination of antibody class/subclass.

### [V] Preparation of Monoclonal Antibody

The method for obtaining a monoclonal antibody from the hybridoma is appropriately selected depending on a necessary amount of the monoclonal antibody and the properties of the hybridoma. Examples include a method of obtaining a monoclonal antibody from ascites of the mouse transplanted with the hybridoma, a method of obtaining a monoclonal antibody from a culture supernatant obtained by cell culture, etc. Insofar as the hybridoma can grow in the peritoneal cavity of the mouse, the monoclonal antibody can be obtained at a high concentration of several milligrams per milliliter of the ascites. For the hybridoma which can not grow in vivo, the antibody is obtained from a culture supernatant of the cell culture. The method of obtaining the monoclonal antibody from cell culture is advantageous over a method conducted in vivo in that although the amount of the antibody produced is low, purification of the antibody is easy with less contamination with immunoglobulins and other contaminants contained in the mouse peritoneal cavity.

When the monoclonal antibody is obtained from ascites of the mouse transplanted with the hybridoma, the hybridoma (approx. 106 or more) is transplanted in the peritoneal cavity of BALB/c mouse to which a substance having immunosuppression properties such as pristane (2,6,10,14-tetramethylpentadecane) has been administered, and after about 1 to 3 weeks, the ascites accumulated is collected. In the case of a heterogenic hybridoma (for example, mouse and rat), it is preferable to use a nude mouse, a radiation-treated mouse, etc.

When the antibody is obtained from the cell culture supernatant, culture methods such as a stationary culture method used in maintaining cells, a high-density culture method and a spinner flask culture method are used to culture the hybridoma thereby obtaining a culture supernatant containing the antibody. Because the serum which can be added to the medium contains contaminants such as other antibodies and albumin and thus often makes purification of the monoclonal antibody from the culture complicated, the amount of the serum to be added to the medium is preferably lower. More preferably, the hybridoma is conditioned to a serum-free medium according to the conventional method and cultured in the serum-free medium. By culturing the hybridoma in the serum-free medium, purification of the monoclonal antibody is facilitated.

Purification of the monoclonal antibody from the ascites or culture supernatant can be carried out by using the method known per se. As the immunoglobulin purification method, methods known in the art such as a fractionation with ammonium sulfate or sodium sulfate, a PEG fractionation, an ethanol fractionation, a DEAE ion-exchange chromatographic method and a gel filtration method can be used to easily purify the monoclonal antibody. When the monoclonal antibody is mouse IgG, the antibody can be purified easily by affinity chromatography using a protein A-bound carrier or an anti-mouse immunoglobulin-bound carrier.

### EXAMPLES

Hereinafter, the present invention is described in more detail by reference to the Examples.

### Example 1

### Production of Monoclonal Antibody Directed to SARS-NP

The monoclonal antibody of the present invention is produced through the following steps [I] to [V]. Specifically, [I] an antigen solution containing a recombinant SARS-NP was prepared by genetic engineering techniques, [II] a mouse was immunized with the antigen solution, [III] spleen cells obtained from the immunized mouse were fused with myeloma cells, [IV] a cell producing a specific antibody to SARS-NP was selected from the resulting hybridomas, and [V] this hybridoma was proliferated in the peritoneal cavity of a mouse, and from its ascites, a monoclonal antibody was separated. Hereinafter, these procedures are described in detail.

### [I] Preparation of Antigen Solution

Using gene analysis software BioEdit version 7.0.0 (BioEdit Corporation), the codons used in a cDNA nucleotide sequence for a nucleocapsid protein of SARS TOR2 strain (disclosed in Gen Bank (Accession No. AY274119; Protein ID: AAP41047.1), which are less frequent codons in Escherichia coli were first converted into frequently used codons. Hereinafter, this sequence is called as SARS-NP cDNA (E. coli). In this SARS-NP cDNA (E. coli) (full length 1269 bp), a cDNA fragment of nucleotide 1 to 660 and a cDNA fragment of nucleotide 600 to 1269 both from the 5′-terminal, were synthesized and then ligated to each other using a restriction enzyme to synthesize a full-length 1269-bp SARS-NP cDNA. Then, this synthetic SARS-NP cDNA sequence was used to prepare two kinds of recombinant SARS-NP (GST fusion type and His-tagged type). Hereinafter, the procedure for this preparation is described in detail.

(1) Preparation of a Vector Containing a cDNA Fragment Consisting of the Sequence of Nucleotide 1 to 660

A vector containing a cDNA fragment consisting of the sequence of nucleotide 1 to 660 of the SARS-NP cDNA sequence was prepared by PCR and TA cloning. First, 8 primers (SEQ ID NOS: 3 to 10) were synthesized, and these primers were used in PCR (first PCR). The reaction condition of the first PCR was 16 cycles of 30 seconds at 95° C., 30 seconds at 56.1° C. and 30 seconds at 72° C. The composition of the reaction solution in the first PCR is as follows:

### (First PCR Reaction Solution)

10 μM primer (SEQ ID NO: 3)-containing primer solution: 0.5 μL
10 μM primer (SEQ ID NO: 4)-containing primer solution: 0.5 μL
10 μM primer (SEQ ID NO: 5)-containing primer solution: 0.5 μL
10 μM primer (SEQ ID NO: 6)-containing primer solution: 0.5 μL
10 μM primer (SEQ ID NO: 7)-containing primer solution: 0.5 μL
10 μM primer (SEQ ID NO: 8)-containing primer solution: 0.5 μL
10 μM primer (SEQ ID NO: 9)-containing primer solution: 0.5 μL
10 μM primer (SEQ ID NO: 10)-containing primer solution: 0.5 μL
2.5 mM dNTP solution (Takara Bio): 1.6 μL

### 1.5 U/μL Ex-Taq (Takara Bio): 0.1 μL

10×Ex-Taq buffer (Takara Bio): 2 μL
Distilled water: 12.3 μL

When the first PCR was finished, the reaction solution was used in the second PCR. The reaction condition of the second PCR was 30 cycles of 30 seconds at 95° C., 30 seconds at 58.5° C. and 30 seconds at 72° C. The composition of the reaction solution in the second PCR is as follows:

### (Second PCR Reaction Solution)

Reaction solution after the first PCR reaction: 1 μL
10 μM primer (SEQ ID NO: 11)-containing primer solution: 0.5 μL
10 μM primer (SEQ ID NO: 12)-containing primer solution: 0.5 μL
2.5 mM dNTP solution (Takara Bio): 1.6 μL

### 1.5 U/μL Ex-Taq (Takara Bio): 0.1 μL

10×Ex-Taq buffer (Takara Bio): 2 μL
Distilled water: 14.3 μL

When the second PCR was finished, 4 μL of the reaction solution was used in TA cloning to prepare a vector containing the cDNA fragment consisting of the sequence of nucleotide 1 to 660. In TA cloning, TOPO TA Cloning kit (Invitrogen) was used. Vector pCR TOPO (1-660) containing the cDNA fragment consisting of the sequence of nucleotide 1 to 660 was thereby obtained.

(2) Preparation of a Vector Containing a cDNA Fragment Consisting of the Sequence of Nucleotide 600 to 1269

A vector containing a cDNA fragment consisting of the sequence of nucleotide 600 to 1269 of the SARS-NP cDNA sequence was prepared in the same manner as described in the above (1). Eight primers (SEQ ID NOs: 13 to 20) were synthesized, and these primers were used in PCR (first PCR). The reaction condition of the first PCR was 16 cycles of 30 seconds at 95° C., 30 seconds at 55.1° C. and 30 seconds at 72° C. The composition of the reaction solution in the first PCR is the same as in the above (1) except that the primer solutions were replaced by solutions each containing the primers of SEQ ID NOs: 13 to 20, respectively. Following the first PCR, the second PCR and TA cloning were carried out in the same manner as in (1) above, and finally, the vector pCR TOPO (600-1269) containing a cDNA fragment consisting of the sequence of nucleotide 600 to 1269 was obtained.

(3) Preparation of a Vector Containing the Full-Length SARS-NP cDNA

The pCR TOPO (1-660) and pCR TOPO (600-1269) were used to prepare a vector containing the full-length 1269-bp SARS-NP cDNA. First, pCR TOPO (1-660) was treated with restriction enzymes SmaI and HindIII to prepare a cDNA fragment containing the vector region and the cDNA fragment containing the sequence of nucleotide 1 to 660 from pCR TOPO (1-660). Similarly, pCR TOPO (600-1269) was treated with restriction enzymes SmaI and HindIII to prepare a cDNA fragment containing the sequence of nucleotide 600 to 1269 from pCR TOPO (600-1269). Then, the cDNA fragment containing the vector region and the cDNA fragment containing the sequence of nucleotide 1 to 660, the cDNA fragment containing the sequence of nucleotide 600 to 1269, and a DNA Ligation Kit Ver 2.1 (Takara Bio) were used to prepare the vector pCR TOPO (1-1269) containing the full-length 1269-bp SARS-NP cDNA. The nucleotide sequence of the SARS-NP cDNA contained in pCR TOPO (1-1269) is shown in SEQ ID NO: 1. The comparison between this nucleotide sequence and the nucleotide sequence of the SARS-NP cDNA (E. coli) reveals that the nucleotide 675 was changed from G to A, and the nucleotide 1107 was changed from C to A, but the amino acids encoded by these nucleotide sequences were the same as those located in TOR2SARS-NP cDNA (E. coli). An amino acid sequence predicted from the nucleotide sequence of the SARS-NP cDNA set forth in SEQ ID NO: 1 is shown in SEQ ID NO: 2.

### (4) Preparation of Gst-Fused Recombinant SARS-NP

GST-fused E. coli recombinant protein expression vector pGEX-2TK (Amersham Bioscience) was treated with restriction enzymes EcoRI and BamHI followed by with alkaline phosphatase to prepare a 4.9-kbp vector fragment. Similarly, pCR TOPO (1-1269) containing the full-length 1269-bp SARS-NP cDNA was treated with restriction enzymes EcoRI and BamHI to prepare a DNA fragment containing the full-length 1269-bp SARS-NP cDNA. Then, the 4.9 kbp vector fragment, the SARS-NP cDNA-containing DNA fragment and a DNA Ligation Kit Ver 2.1 (Takara Bio) were used to prepare E. coli expression vector pGEX-2TK (SARS-NP) containing the full-length 1269-bp SARS-NP cDNA.

Then, Escherichia coli containing the E. coli expression vector pGEX-2TK (SARS-NP) was cultured in an LB medium. In the logarithmic phase after culture was initiated, IPTG was added at a final concentration of 1 mM to the E. coli culture, and the Escherichia coli was cultured at room temperature for 18 hours. After completion of culture, the E. coli cells were recovered and suspended in PBS (1% Triton X). In the suspension, the Escherichia coli cells were disrupted with a sonicator, and the precipitated fraction was washed with 100 mM Tris buffer, pH 8.0 (150 mM NaCl) and then lysed in 100 mM Tris buffer, pH 8.0 (8 M urea, 150 mM KCl). The resulting solution was used as a GST-fused recombinant SARS-NP antigen solution.

### (5) Preparation of His-Tagged Recombinant SARS-NP

Vector pCDNA 3.1 (Invitrogen) was treated with restriction enzymes EcoRI and BamHI followed by with alkaline phosphatase to prepare a 5.0-kbp vector fragment. Similarly, the E. coli expression vector pGEX-2TK (SARS-NP) obtained in (4) above was treated with restriction enzymes EcoRI and BamHI to prepare a DNA fragment containing the full-length 1269-bp SARS-NP cDNA. Then, the 5.0 kbp vector fragment, the SARS-NP cDNA-containing DNA fragment and a DNA Ligation Kit Ver 2.1 (Takara Bio) were used to prepare pCDNA 3.1 (SARS-NP) containing the full-length 1269-bp SARS-NP cDNA.

Subsequently, pCDNA 3.1 (SARS-NP) was treated with restriction enzymes BamHI and XhoI to prepare a DNA fragment containing the full-length 1269-bp SARS-NP cDNA. Similarly, E. coli expression vector pQE30 (Qiagen) was treated with restriction enzymes BamHI and XhoI to prepare a 3.4-kbp vector fragment. Then, the 3.4-kbp vector fragment, the SARS-NP cDNA-containing DNA fragment and a DNA Ligation Kit Ver 2.1 (Takara Bio) were used to prepare E. coli expression vector pQE30 (SARS-NP) containing the full-length 1269-bp SARS-NP cDNA.

Then, Escherichia coli containing the E. coli expression vector pQE30 (SARS-NP) was cultured in an LB medium containing 100 μg/mL ampicillin. In the logarithmic phase after culture was initiated, IPTG was added at a final concentration of 1 mM to the E. coli culture, and the Escherichia coli was cultured for 3.5 hours. After completion of culture, the Escherichia coli was recovered, then suspended in 30 mL of 20 mM sodium phosphate buffer, pH 7.4 (0.5 M NaCl, 1 mM DTT, 1 mg/mL Pefablock (protease inhibitor), 20 mM imidazole), then sonicated (2 minutes×7 times) on ice, and centrifuged to give a soluble fraction which was then applied onto a His-Trap HP column (QIAGEN) to purify the His-tagged recombinant SARS-NP. 20 mM sodium phosphate buffer, pH 7.4, containing this His-tagged recombinant SARS-NP was used as the His-tagged recombinant SARS-NP antigen solution.

### [II] Immunization Step

As the animal to be immunized, Balb/c mice (8-week-old female) were used. The GST-fused recombinant SARS-NP antigen solution obtained in (4) in the above-mentioned [I] and the Hi-tagged recombinant SARS-NP antigen solution obtained in (5) in the above-mentioned [I] were used to immunize Balb/c mice in the following schedule.

(1) A mixture of FCA and the GST-fused recombinant SARS-NP antigen solution (the final concentration of the GST-fused recombinant SARS-NP: 50 μg) was administered intraperitoneally to the mice.
(2) Two weeks thereafter, a mixture of RIBI and the GST-fused recombinant SARS-NP antigen solution (the final concentration of the GST-fused recombinant SARS-NP: 50 μg) was administered intraperitoneally to the mice.
(3) Three weeks thereafter, a mixture of RIBI and the GST-fused recombinant SARS-NP antigen solution (the final concentration of the GST-fused recombinant SARS-NP: 50 μg) was administered intraperitoneally to the mice.
(4) Three weeks thereafter, a mixture of RIBI and the His-tagged recombinant SARS-NP antigen solution (the final concentration of the His-tagged recombinant SARS-NP: 50 μg) was administered intraperitoneally to the mice.
(5) Three weeks thereafter, a mixture of RIBI and the His-tagged recombinant SARS-NP antigen solution (the final concentration of the His-tagged recombinant SARS-NP: 50 μg) was administered intraperitoneally to the mice.
(6) Three weeks thereafter, the His-tagged recombinant SARS-NP antigen solution (the final concentration of the His-tagged recombinant SARS-NP: 50 μg) was injected to the tail vein.

### [III] Cell Fusion Step

In this step, spleen cells obtained form the immunized animal are fused with myeloma cells to prepare a hybridoma. The spleen cells were obtained from the Balb/c mice 3 days after the immunization treatment in (6) in [II] above. The myeloma cells used were X63 cells obtained from a Balb/c mouse myeloma-derived cultured cell line (cell strain X63). For cell fusion, the spleen cells and X63 cells were suspended at a mixing ratio of 7.5:1 in an RPMI-1640 culture medium containing about 50% of polyethylene glycol 4000 (SIGMA) and then incubated. Thereafter, the spleen cells were suspended at a density of 2.5 million cells/ml in a culture medium consisting of an HT medium (RPMI-1640 culture medium supplemented with 10% inactivated fetal serum, 0.1 mM hypoxanthine and 0.016 mM thymidine) and a cloning medium (Sanko Junyaku Co., Ltd.) at the ratio of 1:1, and then added to each well of a 96-well plate (Corning Inc.) to culture the cells.

### [IV] Selection of Hybridoma

The cells after the fusion procedure were cultured in a selective medium and selected for hybridoma. On the day after the fusion procedure, a HAT medium was added to each well of the 96-well microplate to which the cells had been added, and then the cells were cultured. Four, six and eight days after cell fusion, a HT medium was added and the cells were cultured therein, and a well on which a colony of hybridomas grew was confirmed.

### (1) Examination of Reactivity to SARS-NP

Then, the reactivity to SARS-NP of the monoclonal antibody produced by the hybridoma was examined by ELISA with a plate to which the recombinant SARS-NP was immobilized. First, the His-tagged recombinant SARS-NP was immobilized on an ELISA plate. A culture supernatant of the hybridoma was added to, and allowed to react, and then each well was added with a peroxidase-labeled goat anti-mouse antibody followed by with a peroxidase substrate solution to develop color and its absorbance was measured. Thirty hybridomas (Hybridoma Nos. 1 to 30) producing monoclonal antibodies having strong reactivity to the His-tagged recombinant SARS-NP were thereby obtained.

### (2) Study of Epitopes of the Monoclonal Antibodies

The amino acid sequence of SARS-NP was divided into 3 regions, that is, Region A (1-141), Region B (142-282) and Region C (283-422) as shown in FIG. 3, and which region contained an epitope recognized by each monoclonal antibody was examined.

In this study, two recombinant proteins were used. In preparing each of the recombinant proteins, a cDNA fragment encoding an amino acid sequence of amino acid 1 to 282 and a cDNA fragment encoding an amino acid sequence of amino acid 142 to 422 both from the N-terminal of the amino acid sequence of SARS-NP (sequence of amino acid 1 to 422), were synthesized by using, as a template, the E. coli expression vector pGEX-2TK (SARS-NP) obtained in (4) in the above-mentioned [I]. By using the synthesized cDNA fragments, a recombinant protein (SARS-NP N-terminal protein) corresponding to the amino acid sequence of amino acid 1 to 282 and a recombinant protein (SARS-NP C-terminal protein) corresponding to the amino acid sequence of amino acid 142 to 422, in the SARS-NP amino acid sequence, were obtained. Hereinafter, the method of obtaining the recombinant proteins is described in detail.

Preparation of a Vector Containing a cDNA Fragment Encoding the Amino Acid Sequence of Amino Acid 1 to 282

PCR was carried out using E. coli expression vector pGEX-2TK (SARS-NP) and primers of SEQ ID NOs: 11 and 21. The reaction condition of PCR was 30 cycles of 30 seconds at 95° C., 30 seconds at 58.5° C. and 30 seconds at 72° C. The composition of the PCR reaction solution is as follows:

10 μg/mL E. coli expression vector pGEX-2TK (SARS-NP): 1 μL
10 μM primer (SEQ ID NO: 11)-containing primer solution: 0.5 μL
10 μM primer (SEQ ID NO: 21)-containing primer solution: 0.5 μL
2.5 mM dNTP solution (Takara Bio): 1.6 μL

### 1.5 U/EL Ex-Taq (Takara Bio): 0.1 μL

10×Ex-Taq buffer (Takara Bio): 2 μL
Distilled water: 14.3 μL

After the PCR was finished, 4 μL of the reaction solution was used in TA cloning to prepare a vector containing a cDNA fragment corresponding to the region of nucleotide 1 to 846 of the full-length 1269-bp SARS-NP cDNA. In TA cloning, a TOPO TA Cloning kit (Invitrogen) was used. Vector pCR N (1-846) containing a cDNA fragment corresponding to the region of nucleotide 1 to 846 was thereby obtained.

### Preparation of a Vector Containing a cDNA Fragment Encoding the Amino Acid Sequence of Amino Acid 142 to 422

PCR was carried out using E. coli expression vector pGEX-2TK (SARS-NP) and primers of SEQ ID NOs: 22 and 23. The reaction conditions etc. of PCR were the same as in preparation of the vector containing a cDNA fragment encoding the amino acid sequence of amino acid 1 to amino acid 282. Vector pCR C (427-1269) containing a cDNA fragment corresponding to the region of nucleotide 427 to 1269 in the SARS-NP cDNA sequence was thereby obtained.

### Preparation of SARS-NP N-Terminal Protein

The above-mentioned pCR N (1-846) was treated with a restriction enzyme BamHI to prepare a 5.0-kbp cDNA fragment. Similarly, E. coli expression vector pQE30 (Qiagen) was treated with restriction enzymes BamHI and XhoI to prepare a 3.4-kbp vector fragment. Then, the 3.4 kbp vector fragment, the above 5.0-kbp cDNA fragment and a DNA Ligation Kit Ver 2.1 (Takara Bio) were used to prepare E. coli expression vector pQE30 N (1-846) containing a cDNA fragment corresponding to the sequence of nucleotide 1 to 846.

Then, the E. coli expression vector pQE30 N (1-846) was introduced into Escherichia coli which was then cultured in an LB medium containing 100 μg/mL ampicillin. In the logarithmic phase after culture was initiated, IPTG was added at a final concentration of 1 mM to the E. coli culture, and the Escherichia coli was cultured for 3.5 hours. After completion of culture, the Escherichia coli was recovered, then suspended in 30 mL of 20 mM sodium phosphate buffer, pH 7.4 (0.5 M NaCl, 1 mM DTT, 1 mg/mL Pefablock (protease inhibitor), 20 mM imidazole), sonicated (2 minutes×7 times) on ice, and centrifuged to give a soluble fraction which was applied onto a His-Trap HP column (QIAGEN) to purify the SARS-NP N-terminal protein.

### Preparation of SARS-NP C-Terminal Protein

The above-mentioned pCR C (427-1269) was used, and SARS-NP C-terminal protein was purified in the same manner as described in the above purification of the His-tagged recombinant SARS-NP N-terminal protein.

Each of the resulting proteins (SARS-NP N-terminal protein and SARS-NP C-terminal protein) was immobilized on an ELISA plate. A culture supernatant of each of the hybridomas (Nos. 1 to 30) was added to, and reacted with, each plate, and then each well was added with a peroxidase-labeled goat anti-mouse antibody followed by a peroxidase substrate solution to develop color and its absorbance was measured. It was thereby revealed that the hybridoma showing reactivity to only the SARS-NP N-terminal protein produces a monoclonal antibody recognizing an epitope located in the Region A in FIG. 3, the hybridoma showing reactivity to only the SARS-NP C-terminal protein produces a monoclonal antibody recognizing an epitope located in the Region C in FIG. 3, and the hybridoma showing reactivity to both the proteins produces a monoclonal antibody recognizing an epitope located in the Region B in FIG. 3.

The results (absorbance) in (1) and (2) above are collectively shown in Table 1.

TABLE 1

| | Region | Reactivity | | |
|---|---|---|---|---|
| | where | His-tagged | SARS-NP | SARS-NP |
| | epitope | recombinant | N-terminal | C-terminal |
| Hybridoma | exists | SARS-NP | protein | protein |
| No. 1 | A | 3.955 | 3.955 | 0.138 |
| No. 2 | | 3.267 | 3.529 | 0.011 |
| No. 3 | | 3.252 | 3.614 | 0.036 |
| No. 4 | | 3.431 | 3.460 | 0.012 |
| No. 5 | | 3.308 | 3.957 | 0.015 |
| No. 6 | | 3.522 | 3.619 | 0.015 |
| No. 7 | | 2.979 | 3.667 | 0.021 |
| No. 8 | | 2.663 | 3.492 | 0.049 |
| No. 9 | | 2.804 | 3.619 | 0.011 |
| No. 10 | | 3.543 | 3.737 | 0.012 |
| No. 11 | | 2.687 | 3.496 | 0.011 |
| No. 12 | B | 3.554 | 3.679 | 1.986 |
| No. 13 | | 2.955 | 3.151 | 2.444 |
| No. 14 | C | 1.914 | 0.014 | 2.617 |
| No. 15 | | 2.073 | 0.008 | 3.096 |
| No. 16 | | 3.628 | 0.121 | 3.639 |
| No. 17 | | 2.906 | 0.053 | 3.512 |
| No. 18 | | 3.448 | 0.051 | 3.550 |
| No. 19 | | 3.318 | 0.031 | 3.537 |
| No. 20 | | 3.314 | 0.031 | 3.541 |
| No. 21 | | 3.438 | 0.048 | 4.009 |
| No. 22 | | 2.849 | 0.062 | 3.423 |
| No. 23 | | 3.401 | 0.083 | 3.415 |
| No. 24 | | 2.452 | 0.042 | 3.367 |
| No. 25 | | 3.390 | 0.061 | 3.662 |
| No. 26 | | 3.169 | 0.037 | 3.573 |
| No. 27 | | 3.365 | 0.061 | 3.592 |
| No. 28 | | 3.045 | 0.028 | 3.438 |
| No. 29 | | 3.557 | 0.077 | 3.511 |
| No. 30 | | 2.460 | 0.032 | 4.002 |

### [V] Preparation of Monoclonal Antibodies

Among the hybridomas in Table 1 above, the monoclonal antibodies produced by hybridoma Nos. 1, 2, 3, 12, 13, 14, 15, 16 and 17 were purified. In purification, each hybridoma was transplanted in the peritoneal cavity of a BALB/c mouse, and 10 days later, an ascites accumulated was collected. From the collected ascites, monoclonal antibody was purified with a protein A column Hyper D (Perseptive Biosystems). By this operation, 9 monoclonal antibodies (Monoclonal Antibody Nos. 1, 2, 3, 12, 13, 14, 15, 16 and 17) were obtained.

### Example 2

### Application to Immunochromatography

Immunochromatography was carried out using the 9 monoclonal antibodies (Monoclonal Antibody Nos. 1, 2, 3, 12, 13, 14, 15, 16 and 17) obtained in Example 1.

### Preparation of a Test Device for Immunochromatography

In this example, a test device in the form as shown in FIG. 2 was used. In the test device in this example, a backing sheet having an adhesive surface was used as a substrate 1; Whatman WF 1.5, as an adsorbent member 5; and a nitrocellulose membrane as a chromatographic carrier 4. The chromatographic carrier 4 has a judging part 6 to which one of the 9 monoclonal antibodies described above has been immobilized. In this example, the sample addition member of the test device is dipped in a measurement sample prepared from a sample, thereby developing the sample solution via the capillary phenomenon toward the judging part.

### Antibody-Sensitized Latex

One of the 9 monoclonal antibodies was immobilized on blue polystyrene latex particles (average particle diameter 0.3 μm), and the blue polystyrene latex particles were suspended at a density of 0.2% (w/v) in 10 mM phosphate buffer, pH 8.0, and used as antibody-sensitized latex solution.

### Preparation of a Sample

The His-tagged recombinant SARS-NP obtained in (5) in [I] above was dissolved at a concentration of 18.2 ng/mL in a sample buffer in POCTEM (Sysmex Corporation) and used as a positive sample. A sample buffer not containing the His-tagged recombinant SARS-NP was used as a negative sample.

### Measurement Method

20 μL of a sample, 25 μL of an extraction solution in POCTEM (Sysmex Corporation), and 30 μL of the antibody-sensitized latex were mixed to prepare a measurement sample. The test device was introduced into a tube containing this measurement sample such that the sample addition member 2 was dipped in the measurement sample, and then left at room temperature for 20 minutes, and a blue band appeared on the judging part 6 in the chromatographic carrier 4 was observed.

The measurement results are shown in Table 2. Each band was judged depending on the degree of blue coloration into 5 levels: “−”, “W”), “1+”, “2+” and “3+”. In the Table, “No.” indicates Monoclonal Antibody Number, and A, B and C represent the region in which the epitope for each antibody is present. Whether an non-specific reaction had occurred or not was confirmed in every case by measuring the His-tagged recombinant SARS-NP-free sample buffer as a negative sample. In the table, ▴ indicates a sample in which an non-specific reaction was observed.

TABLE 2

| | | Chromatographic carrier | | | | | | | | |
|---|---|---|---|---|---|---|---|---|---|---|
| | | A | | | B | | C | | | |
| Blue latex particles | | No. 1 | No. 2 | No. 3 | No. 12 | No. 13 | No. 14 | No. 15 | No. 16 | No. 17 |
| A | No. 1 | — | — | — | — | — | 3+ | 2+ | 1+ | 2+ |
| | No. 2 | — | — | — | — | ▴1+ | 3+ | 2+ | 1+ | 2+ |
| | No. 3 | — | — | — | — | — | 3+ | 1+ | 1+ | 1+ |
| B | No. 12 | — | — | — | — | — | 3+ | 2+ | 1+ | 2+ |
| | No. 13 | 2+ | 2+ | 1+ | ▴3+ | ▴2+ | 3+ | 2+ | 1+ | 1+ |
| C | No. 14 | 2+ | 2+ | 1+ | 3+ | — | — | — | — | — |
| | No. 15 | 1+ | 1+ | 1+ | 2+ | — | — | — | — | — |
| | No. 16 | 2+ | 2+ | 1+ | 3+ | — | 1+ | 1+ | — | — |
| | No. 17 | W | W | 1+ | 1+ | — | — | — | — | — |

“−”, “W”, “1+”, “2+” and “3+” were established under the criteria in Table 3 which are based on the measurements of appeared bands with TRS 3000 Membrane Strip Reader (Bio Rad). In the Table, the ROD value is a value obtained by subtracting the measured density of a nitrocellulose membrane as background from the measured density of a band. A blue band assigned “W”, “1+”, “2+” or “3+” can be visually confirmed.

TABLE 3

| | ROD value |
|---|---|
| — | 0.000-0.006 |
| W | 0.007-0.014 |
| 1+ | 0.015-0.029 |
| 2+ | 0.030-0.079 |
| 3+ | 0.080 or more |

As can be seen from Table 2, the combination of the antibody immobilized on the chromatographic carrier and the antibody labeled with the colored latex in immunochromatography is preferably a combination of the antibodies judged to be “1+”, more preferably a combination of the antibodies judged to be “2+”, and most preferably a combination of the antibodies judged to be “3+”. Three hybridomas (Hybridoma Nos. 2, 12 and 14) producing the monoclonal antibodies (Monoclonal Antibody Nos. 2, 12 and 14) which showed high sensitivity of the measurement in this example were deposited in the International Patent Organism Depositary in National Institute of Advanced Industrial Science and Technology (Tsukuba Central 6, 1-1-1 Higashi, Tsukuba, Ibaraki, Japan) on Feb. 15, 2005. The accession number of each hybridoma is as follows:

### Accession Number of Hybridoma No. 2: FERM ABP-10678

### Accession Number of Hybridoma No. 12: FERM ABP-10679

### Accession Number of Hybridoma No. 14: FERM ABP-10680

Now, the monoclonal antibodies which recognize the epitope located in the Region A are classified into group A antibody, the monoclonal antibodies which recognize the epitope located in the Region B are classified into group B antibody, and the monoclonal antibodies which recognize the epitope located in the Region C are classified into group C antibody. From Table 2, it was found that the high sensitivity of the measurement can be obtained by using the group C antibody as the antibody immobilized on the chromatographic carrier or labeled with colored latex.

It was found that relatively high sensitivity of the measurement can be obtained when the combination of the antibodies immobilized on the chromatographic carrier and labeled with colored latex is a combination of the group A antibody and the group C antibody or a combination of the group B antibody and the group C antibody.

It was also found that high sensitivity of the measurement can be obtained by using the group A or C antibody as the antibody immobilized on the chromatographic carrier. It was further found that high sensitivity of the measurement can be obtained where the group A antibody immobilized on the chromatographic carrier is combined with the group C antibody as the antibody labeled with colored latex. It was also found that high sensitivity of the measurement can be obtained where the group C antibody immobilized on the chromatographic carrier is combined with the group A or B antibody as the antibody labeled with colored latex. It was also found that relatively high sensitivity of the measurement can be obtained where the monoclonal antibody No. 12 belonging to the group B used as the antibody immobilized on the chromatographic carrier is combined with the group C antibody as the antibody labeled with colored latex.

The above preferable combinations of the antibodies are not limited to those for use in immunochromatography and can be applied to other immunological measurement methods.

### Example 3

### Application to ELISA

ELISA was carried out using the monoclonal antibody Nos. 1 and 14 obtained in Example 1. In this example, the monoclonal antibody No. 14 was immobilized on an ELISA plate, while the monoclonal antibody No. 1 was labeled with alkaline phosphatase. The His-tagged recombinant SARS-NP obtained in (5) in [I] above was dissolved at concentrations of 0, 0.195, 0.39, 0.78, 1.56, and 3.12 ng/mL in 10 mM phosphate buffer, pH 7.0 and used as samples.

For measurement, 100 μL of the sample was first added to the ELISA to which the monoclonal antibody No. 12 was immobilized, and stirred at room temperature for 30 minutes. The plate was washed with 10 mM phosphate buffer, pH 7.0, and then 100 μL of the His-tagged recombinant SARS-NP antigen solution (20 ng/mL) obtained in Example 1 was added thereto and stirred at room temperature for 30 minutes. After the plate was washed with the phosphate buffer, 100 μL of 10 mM phosphate buffer, pH 7.0, containing 5 U/mL alkaline phosphatase-labeled monoclonal antibody No. 1 was added thereto and stirred at room temperature for 30 minutes. Then, the plate was washed, the color was developed with 100 μL of the peroxidase substrate solution and its absorbance was measured.

As a comparative example, two antibodies used in the commercial product SARS-Nucleocapsid Active ELISA (IMGENEX) were used and subjected in the same manner to ELISA.

The results are shown in FIG. 4. In this figure, the absorbance (OD405/OD655) is shown on the x-axis, and the concentration (ng/mL) of the His-tagged recombinant SARS-NP contained in a sample is shown on the y-axis. The solid line shows the results where the monoclonal antibodies Nos. 1 and 14 were used, and the dotted line shows the results where the antibodies in the commercial product were used.

In FIG. 4, very strong signal intensity was shown when the monoclonal antibodies Nos. 1 and 14 were used. From this result, it was found that when the monoclonal antibodies Nos. 1 and 14 are used in ELISA, measurement can be carried out with very high sensitivity.

Immunochromatography was carried out using the antibodies (monoclonal antibodies Nos. 1 and 14) and the samples (His-tagged recombinant SARS-NP at concentrations of 0, 0.195, 0.39, 0.78, 1.56, and 3.12 ng/mL) used in Example 3. The results are shown in Table 4. The immunochromatography was carried out in the same manner as in Example 2. In this example, the monoclonal antibody No. 14 was immobilized on the chromatographic carrier, and the monoclonal antibody No. 1 was labeled with colored latex.

TABLE 4

| Concentration of | |
|---|---|
| SARS-NP | |
| in sample (ng/mL) | Judgment |
| 0 | — |
| 0.195 | W |
| 0.39 | 1+ |
| 0.78 | 1+ |
| 1.56 | 2+ |
| 3.12 | 2+ |

Table 4 shows that the judgment result “W” was obtained when the concentration of SARS-NP in the sample was 0.195 ng/mL. From this result, it was found that when the monoclonal antibodies Nos. 1 and 14 are used in immunochromatography, SARS-NP can be detected insofar as the concentration of SARS-NP in the sample is at least 0.195 ng/mL.

### Example 4

### Study of Combination of Antibodies in ELISA

ELISA was carried out using the monoclonal antibodies Nos. 1, 3, 8, 12, 14, 15, 16 and 23 obtained in Example 1. Each of these monoclonal antibodies was immobilized on an ELISA plate and labeled with biotin, and which combination of the monoclonal antibodies could provide highly sensitive detection in ELISA was studied.

Each of the monoclonal antibodies was diluted to 1 μg/mL with 0.1 M phosphate buffer, pH 7.5 containing 0.1% sodium azide. The antibody solution (100 μL) was added to each well of the ELISA plate and left overnight at 4° C. The plate was washed with buffer II (10 mM sodium phosphate, 150 mM-NaCl, 0.05% Tween 20) in a plate washing machine. 300 μL of buffer I (10 mM sodium phosphate, pH 7.0, 2.5 mM EDTA, 1% BSA, 150 mM NaCl) was added to each well and left overnight at 4° C. 100 μL of the His-tagged recombinant SARS-NP antigen solution (20 ng/mL) obtained in Example 1 and diluted with buffer I was added to the plate to which the antibody was thus immobilized, and then stirred at room temperature for 30 minutes. The wells were washed with buffer II, and 0.5 μg/mL biotin-labeled monoclonal antibody diluted with buffer I was added thereto and stirred at room temperature for 30 minutes. Each well was washed with buffer II, and 100 μL of peroxidase-labeled streptavidin diluted with buffer I (20 mU/mL) was added thereto and stirred at room temperature for 30 minutes. Each well was washed with buffer II, and 100 μL of the peroxidase substrate solution was added thereto and stirred at room temperature for 2.5 minutes, followed by adding 100 μL of 2 N sulfuric acid. The absorbance (OD492/OD690) of the plate was measured. One sample was measured on two wells to determine their average absorbance. The blank obtained by measuring the antigen-free antigen solution (that is, the 0 ng/mL SARS-NP solution) was subtracted from the above average absorbance, to give the absorbance as the result.

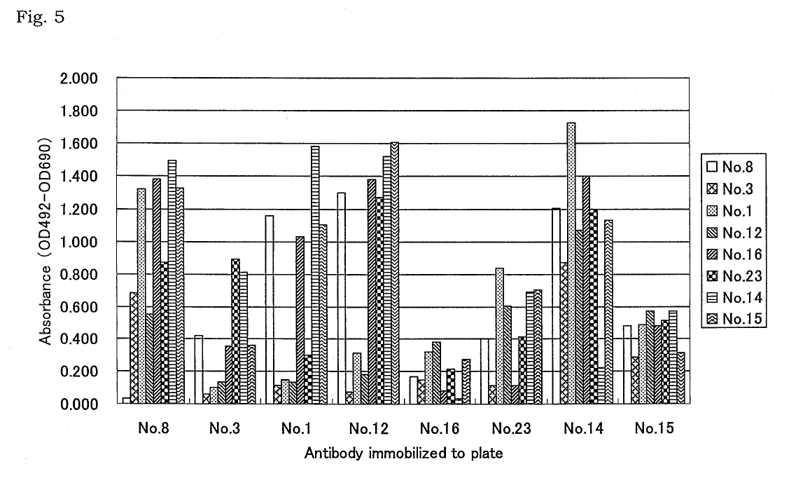

The results are shown in FIG. 5. In this figure, the absorbance (OD492/OD690) is shown on the x-axis, and the antibody immobilized on the plate is shown on the y-axis.

As can be seen from FIG. 5, the combination of the antibody immobilized on the ELISA plate and the antibody labeled with biotin in ELISA is preferably a combination of the antibodies giving an absorbance (OD492/OD690) of 1.000 or more, more preferably a combination of the antibodies giving an absorbance of 1.200 or more, and most preferably a combination of the antibodies giving an absorbance of 1.450 or more as judgment. Two hybridomas (hybridomas No. 1 and 15) producing the monoclonal antibodies No. 1 and 15, among the 4 monoclonal antibodies (monoclonal antibodies Nos. 1, 12, 14 and 15) giving highly sensitive measurement results in this example, were deposited in the International Patent Organism Depositary in National Institute of Advanced Industrial Science and Technology (Tsukuba Central 6, 1-1-1 Higashi, Tsukuba, Ibaraki, Japan) on Sep. 26, 2006. The accession number of each hybridoma is as follows:

### Accession Number of Hybridoma No. 1: FERM ABP-10687

### Accession Number of Hybridoma No. 15: FERM ABP-10686

Here, the monoclonal antibodies which recognize the epitope located in the Region A is classified as group A antibody; the monoclonal antibodies which recognize the epitope located in the Region B, as group B antibody; and the monoclonal antibodies which recognize the epitope located in the Region C, as group C antibody. From FIG. 5, it was found that when the group C antibody is used as the antibody immobilized on the ELISA plate or labeled with biotin, relatively high sensitivity of the measurement can be obtained.

It was found that relatively high sensitivity of the measurement can be obtained when the combination of the antibody immobilized on the ELISA plate and the antibody labeled with biotin is a combination of the group A antibody and the group C antibody or a combination of the group B antibody and the group C antibody.

It was also found that high sensitivity of the measurement can be obtained where the group A antibody used as the antibody immobilized on the ELISA plate is combined with the group C antibody as the antibody labeled with biotin. It was also found that high sensitivity of the measurement can be obtained where the group B antibody used as the antibody immobilized on the ELISA plate is combined with the group C antibody as the antibody labeled with biotin. It was also found that relatively high sensitivity of the measurement can be obtained where the monoclonal antibody No. 14 belonging to the group C used as the antibody immobilized on the ELISA plate is combined with the group A antibody as the antibody labeled with biotin.

### Example 5

### Measurement of Antigen at Various Concentrations by ELISA

In consideration of the above results, ELISA was carried out in the same manner as in Example 4 except that the combinations of the monoclonal antibodies shown in Table 5 were used. The His-tagged recombinant SARS-NP antigen was used at concentrations of 0 to 3.125 μg/mL diluted in buffer I. The concentration of the biotin-labeled monoclonal antibody was 1 μg/mL, and the time of reaction with the peroxidase substrate solution was 10 minutes.

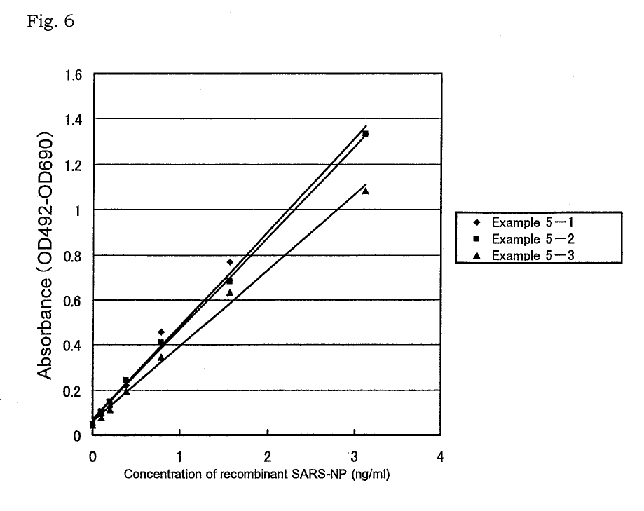

One sample was measured on 4 wells to obtain their average absorbance as the result. The results are shown in FIG. 6.

TABLE 5

| | Antibody immobilized | Biotin-labeled |
|---|---|---|
| Examples | to plate | antibody |
| 5-1 | No. 14 | No. 1 |
| 5-2 | No. 12 | No. 15 |
| 5-3 | No. 12 | No. 14 |

As can be seen from FIG. 6, the absorbance is increased in proportion to the concentration of SARS-NP in the sample. It was thus found that the concentration of SARS can be measured by ELISA using the combination of the antibodies in this example.

The entire disclosure of Japanese Patent Application No. 2005-296542 filed on Oct. 11, 2005 including the specification, claims, drawings and abstract, is incorporated herein by reference.

1. A method for measuring SARS virus nucleocapsid protein (SARS-NP) using first and second antibodies both binding specifically to SARS-NP, wherein the first or second antibody is an antibody recognizing an epitope located in a region (Region C) of amino acid 283 to 422 from the N-terminus of the amino acid sequence of SARS-NP.

2. The method according to claim 1, wherein
the second antibody is the antibody recognizing the epitope located in the Region C,
the first antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP and
the first antibody is immobilized on a solid phase.

3. The method according to claim 1, wherein
the first antibody is the antibody recognizing the epitope located in the Region C,
the second antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP and
the first antibody is immobilized on a solid phase.

4. The method according to claim 1, wherein at least one of the first and second antibodies is a monoclonal antibody.

5. The method according to claim 1, wherein the second antibody is labeled with a labeling substance.

6. The method according to claim 1 which is nephelometric immunoassay, latex agglutination immunoassay, radioimmunoassay, enzyme immunoassay, fluorescent immunoassay, chemiluminescent immunoassay or immunochromatography.

7. A reagent kit for measuring SARS virus nucleocapsid protein (SARS-NP) using first and second antibodies both binding specifically to SARS-NP, comprising a combination of a solid phase to which the first antibody is immobilized and a reagent containing the second antibody labeled with a labeling substance, wherein the first or second antibody is an antibody recognizing an epitope located in a region (Region C) of amino acid 283 to 422 from the N-terminus of an amino acid sequence of SARS-NP.

8. The reagent kit according to claim 7, wherein
the second antibody is the antibody recognizing the epitope located in the Region C, and
the first antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP.

9. The reagent kit according to claim 7, wherein
the first antibody is the antibody recognizing the epitope located in the Region C, and
the second antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP.

10. The reagent kit according to claim 7, wherein at least one of the first and second antibodies is a monoclonal antibody.

11. An immunochromatographic test device for measuring SARS virus using first and second antibodies both binding specifically to SARS virus nucleocapsid protein (SARS-NP), wherein
the first antibody is immobilized on a solid phase, and the second antibody is labeled with a labeling substance,
the immunochromatographic test device comprises a sample addition part to which a measurement sample is added and a sample developing part in which the measurement sample added to the sample addition part is developed; the sample developing part having a judging part to which the first antibody is immobilized and the measurement sample added to the sample addition part being developed toward at least the judging part, and
the first or second antibody is an antibody recognizing an epitope located in a region (Region C) of amino acid 283 to 422 from the N-terminus of the amino acid sequence of SARS-NP.

12. The immunochromatographic test device according to claim 11, wherein
the second antibody is the antibody recognizing the epitope located in the Region C, and
the first antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP.

13. The immunochromatographic test device according to claim 11, wherein
the first antibody is the antibody recognizing the epitope located in the Region C, and
the second antibody is an antibody recognizing an epitope located in a region (Region A) of amino acid 1 to 141 from the N-terminus of the amino acid sequence of SARS-NP or an antibody recognizing an epitope located in a region (Region B) of amino acid 142 to 282 from the N-terminus of the amino acid sequence of SARS-NP.

14. The immunochromatographic test device according to claim 11, wherein at least one of the first and second antibodies is a monoclonal antibody.

15. The immunochromatographic test device according to claim 11, wherein the measurement sample comprises the second antibody labeled with the labeling substrate.

16. The immunochromatographic test device according to claim 11, wherein the sample developing part comprises a label retaining part retaining the second antibody labeled with the labeling substance, and the label retaining part is located upstream from the judging part relative to the direction of the development of the measurement sample toward the judging part.

17. A monoclonal antibody which binds specifically to SARS virus nucleocapsid protein (SARS-NP) and is produced by a hybridoma having an accession number of FERM ABP-10678.

18. A monoclonal antibody which binds specifically to SARS virus nucleocapsid protein (SARS-NP) and is produced by a hybridoma having an accession number of FERM ABP-10679.

19. A monoclonal antibody which binds specifically to SARS virus nucleocapsid protein (SARS-NP) and is produced by a hybridoma having an accession number of FERM ABP-10680.

20. A monoclonal antibody which binds specifically to SARS virus nucleocapsid protein (SARS-NP) and is produced by a hybridoma having an accession number of FERM ABP-10686.

21. A monoclonal antibody which binds specifically to SARS virus nucleocapsid protein (SARS-NP) and is produced by a hybridoma having an accession number of FERM ABP-10687.

22. A hybridoma which is deposited under an accession number of FERM ABP-10678.

23. A hybridoma which is deposited under an accession number of FERM ABP-10679.

24. A hybridoma which is deposited under an accession number of FERM ABP-10680.

25. A hybridoma which is deposited under an accession number of FERM ABP-10686.

26. A hybridoma which is deposited under an accession number of FERM ABP-10687.


SEQUENCE LISTING (23 sequences)

SEQ ID NO: 1 - DNA, 1269 bp, Artificial
  synthetic DNA based on TOR2 SARS-NP cDNA
atgagcgataacggcccgcagagcaaccagcgtagcgcgccgcgtattacctttggcggcccgaccgatagcaccgataacaaccagaacggcggccgtaacggcgcgcgtccgaaacagcgtcgtccgcagggcctgccgaacaacaccgcgagctggtttaccgcgctgacccagcatggcaaagaagaactgcgttttccgcgtggccagggcgtgccgattaacaccaacagcggcccggatgatcagattggctattatcgtcgtgcgacccgtcgtgtgcgtggcggcgatggcaaaatgaaagaactgagcccgcgttggtatttttattatctgggcaccggcccggaagcgagcctgccgtatggcgcgaacaaagaaggcattgtgtgggtggcgaccgaaggcgcgctgaacaccccgaaagatcatattggcacccgtaacccgaacaacaacgcggcgaccgtgctgcagctgccgcagggcaccaccctgccgaaaggcttttatgcggaaggcagccgtggcggcagccaggcgagcagccgtagcagcagccgtagccgtggcaacagccgtaacagcaccccgggcagcagccgtggcaacagcccggcgcgtatggcgagcggcggcggcgaaaccgcgctggcgctgctgctgctagatcgtctgaaccagctggaaagcaaagtgagcggcaaaggccagcagcagcagggccagaccgtgaccaaaaaaagcgcggcggaagcgagcaaaaaaccgcgtcagaaacgtaccgcgaccaaacagtataacgtgacccaggcgtttggccgtcgtggcccggaacagacccagggcaactttggcgatcaggatctgattcgtcagggcaccgattataaacattggccgcagattgcgcagtttgcgccgagcgcgagcgcgttttttggcatgagccgtattggcatggaagtgaccccgagcggcacctggctgacctatcatggcgcgattaaactggatgataaagatccgcagtttaaagataacgtgattctgctgaacaaacatattgatgcgtataaaacctttccgccgaccgaaccaaaaaaagataaaaaaaaaaaaaccgatgaagcgcagccgctgccgcagcgtcagaaaaaacagccgaccgtgaccctgctgccggcggcggatatggatgattttagccgtcagctgcagaacagcatgagcggcgcgagcgcggatagcacccaggcgtaa

SEQ ID NO: 2 - protein, 422 aa, Artificial
  Amino acid sequence deduced from recombinant SARS-NP cDNA
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - DNA, 98 bp, Artificial
  Designed DNA based on TOR2 SARS-NP cDNA
atgagcgataacggcccgcagagcaaccagcgtagcgcgccgcgtattacctttggcggcccgaccgatagcaccgataacaaccagaacggcggccg

SEQ ID NO: 4 - DNA, 100 bp, Artificial
  Designed DNA based on TOR2 SARS-NP cDNA
gtttaccgcgctgacccagcatggcaaagaagaactgcgttttccgcgtggccagggcgtgccgattaacaccaacagcggcccggatgatcagattggc

SEQ ID NO: 5 - DNA, 101 bp, Artificial
  Designed DNA based on TOR2 SARS-NP cDNA
cgcgttggtatttttattatctgggcaccggcccggaagcgagcctgccgtatggcgcgaacaaagaaggcattgtgtgggtggcgaccgaaggcgcgctg

SEQ ID NO: 6 - DNA, 99 bp, Artificial
  Designed DNA based on TOR2 SARS-NP cDNA
cagctgccgcagggcaccaccctgccgaaaggcttttatgcggaaggcagccgtggcggcagccaggcgagcagccgtagcagcagccgtagccgtggc

SEQ ID NO: 7 - DNA, 103 bp, Artificial
  Designed DNA based on TOR2 SARS-NP cDNA
gctgggtcagcgcggtaaaccagctcgcggtgttgttcggcaggccctgcggacgacgctgtttcggacgcgcgccgttacggccgccgttctggttgttatc

SEQ ID NO: 8 - DNA, 102 bp, Artificial
  Designed DNA based on TOR2 SARS-NP cDNA
gataataaaaataccaacgcgggctcagttctttcattttgccatcgccgccacgcacacgacgggtcgcacgacgataatagccaatctgatcatccgggc

SEQ ID NO: 9 - DNA, 101 bp, Artificial
  Designed DNA based on TOR2 SARS-NP cDNA
gtggtgccctgcggcagctgcagcacggtcgccgcgttgttgttcgggttacgggtgccaatatgatctttcggggtgttcagcgcgccttcggtcgccac

SEQ ID NO: 10 - DNA, 101 bp, Artificial
  Designed DNA based on TOR2 SARS-NP cDNA
cagcgcggtttcgccgccgccgctcgccatacgcgccgggctgttgccacggctgctgcccggggtgctgttacggctgttgccacggctacggctgctgc

SEQ ID NO: 11 - DNA, 29 bp, Artificial
  Designed DNA based on TOR2 SARS-NP cDNA
cccggatccatgagcgataacggcccgca

SEQ ID NO: 12 - DNA, 23 bp, Artificial
  Designed DNA based on TOR2 SARS-NP cDNA
cccacagccgtaacagcaccccg

SEQ ID NO: 13 - DNA, 104 bp, Artificial
  Designed DNA based on TOR2 SARS-NP cDNA
acagccgtaacagcaccccgggcagcagccgtggcaacagcccggcgcgtatggcgagcggcggcggcgaaaccgcgctggcgctgctgctgctggatcgtctg

SEQ ID NO: 14 - DNA, 106 bp, Artificial
  Designed DNA based on TOR2 SARS-NP cDNA
aaagcgcggcggaagcgagcaaaaaaccgcgtcagaaacgtaccgcgaccaaacagtataacgtgacccaggcgtttggccgtcgtggcccggaacagacccaggg

SEQ ID NO: 15 - DNA, 103 bp, Artificial
  Designed DNA based on TOR2 SARS-NP cDNA
gcgcagtttgcgccgagcgcgagcgcgttttttggcatgagccgtattggcatggaagtgaccccgagcggcacctggctgacctatcatggcgcgattaaac

SEQ ID NO: 16 - DNA, 100 bp, Artificial
  Designed DNA based on TOR2 SARS-NP cDNA
acctttccgccgaccgaaccgaaaaaagataaaaaaaaaaaaaccgatgaagcgcagccgctgccgcagcgtcagaaaaaacagccgaccgtgaccctgc

SEQ ID NO: 17 - DNA, 104 bp, Artificial
  Designed DNA based on TOR2 SARS-NP cDNA
gctcgcttccgccgcgctttttttggtcacggtctggccctgctgctgctggcctttgccgctcactttgctttccagctggttcagacgatctagcagcagca

SEQ ID NO: 18 - DNA, 102 bp, Artificial
  Designed DNA based on TOR2 SARS-NP cDNA
cgcgctcggcgcaaactgcgcaatctgcggccaatgtttataatcggtgccctgacgaatcagatcctgatcgccaaagttgccctgggtctgttccgggcc

SEQ ID NO: 19 - DNA, 108 bp, Artificial
  Designed DNA based on TOR2 SARS-NP cDNA
ggttcggtcggcggaaaggttttatacgcatcaatatgtttgttcagcagaatcacgttatctttaaactgcggatctttatcatccagtttaatcgcgccatgatag

SEQ ID NO: 20 - DNA, 102 bp, Artificial
  Designed DNA based on TOR2 SARS-NP cDNA
ttacgcctgggtgctatccgcgctcgcgccgctcatgctgttctgcagctgacggctaaaatcatccatatccgccgccggcagcagggtcacggtcggctg

SEQ ID NO: 21 - DNA, 32 bp, Artificial
  Designed DNA based on TOR2 SARS-NP cDNA
ggatccctgttcctgttccgggccacgacggc

SEQ ID NO: 22 - DNA, 26 bp, Artificial
  Designed DNA based on TOR2 SARS-NP cDNA
ggatccaccccgaaagatcatattgg

SEQ ID NO: 23 - DNA, 23 bp, Artificial
  Designed DNA based on TOR2 SARS-NP cDNA
cccttacgcctgggtgctatccg